(19) United States
(12) Patent Application Publication
(10) Pub. No.: US 2024/0261084 A1
(43) Pub. Date: Aug. 8, 2024

(54) SCAFFOLDS FOR BONE-SOFT TISSURE INTERFACE AND METHODS OF FABRICATING THE SAME

(71) Applicant: UNM Rainforest Innovations, Albuquerque, NM (US)

(72) Inventors: Christina Salas, Albuquerque, NM (US); Sherif Hassan Abdelkader Aboubakr, Albuquerque, NM (US); Steven Nery, Albuquerque, NM (US); Christopher Buksa, Albuquerque, NM (US); Alexander Hamilton, Albuquerque, NM (US)

(73) Assignee: UNM Rainforest Innovations, Albuquerque, NM (US)

(21) Appl. No.: 18/609,914

(22) Filed: Mar. 19, 2024

Related U.S. Application Data
(62) Division of application No. 16/632,313, filed on Jan. 17, 2020, now abandoned.
(60) Provisional application No. 62/534,202, filed on Jul. 19, 2017.
(60) Provisional application No. 62/533,604, filed on Jul. 17, 2017.

(51) Int. Cl. A61F 2/08; A61F 2/28; A61F 2/30; B33Y 10/00; B33Y 30/00; B33Y 80/00; D01D 5/00

(52) U.S. Cl. CPC A61F 2/08; A61F 2/28; A61F 2/30756; D01D 5/0061; D01D 5/0076; A61F 2002/30766; A61F 2002/30985; B33Y 10/00; B33Y 30/00; B33Y 80/00

(57) ABSTRACT

A device for regenerating musculoskeletal tissue having a scaffold comprised of fiber layers adapted to provide mechanical integrity to the scaffold in the form of increased tensile and compressive resistance and one or more other layers adapted to provide mechanical integrity and to provide a suitable biochemical environment.

### RELATED APPLICATIONS

This application claims priority to U.S. Provisional Application Ser. Nos. 62/533,604 filed 17 Jul. 2017 and 62/534,020 filed 18 Jul. 2017, both of which are hereby incorporated by reference in their entireties.

### BACKGROUND OF THE INVENTION

Ligament healing after rupture can be delayed months or years and the native (original) tissue (collagen type I) is often replaced through fibroblast synthesis of collagen type III. The replacement scar-like tissue is biomechanically, biochemically, and histologically insufficient to the native tissue. For this reason, surgical intervention in the form of ligament reconstruction is necessary. This procedure requires that a tendon be removed from another region of the body and used as a ligament replacement. For this reconstruction, tunnels are drilled through the bone and the tendon is fed through each tunnel, secured with a bone plug or set screw. There is a stress concentration generated at the tendon/bone interface which may lead to failure of the reconstruction. Unfortunately, these autographs have been shown to cause donor-site morbidity and do not match the mechanical properties of the native ligament. It is also important to note that no current ligament reconstruction procedure can completely restore the kinematics of the pathologic joint. Orthopaedic-related research has therefore, focused on strategies to improve and accelerate the healing process through tissue-engineered scaffolds. An optimal interface scaffold would be tri-phasic, including the bone, ligament (tendon, cartilage, or other soft tissue), and the hard/soft tissue interface region. It would be histologically and biochemically similar to the native tissue and mechanically competent to resist high tensile and compressive joint loads. Additionally, it would minimize the complexity of integration of the soft-tissue into the existing bony anatomy.

The insertions of ligaments onto bone (ligament-bone interface) are specially adapted to allow proper distribution and dissipation of stresses at this interface. However, significant force is transmitted through this interface, so injury and tissue damage most often occur (avulsion). This interface (enthesis) consists of gradients in mechanical properties, chemical properties and cell phenotypes, and structurally continuous over four compositionally distinct regions: ligament, non-mineralized fibrocartilage, mineralized fibrocartilage and bone. The interface exhibits a gradual increase in mineral content with gradual decrease in collagen fiber organization.

In the ligament region, collagen fibers are aligned and parallel, then start to bend and intercross along the interface, and become more disorganized in the bone region. However, collagen content is observed in the ligament and bone regions, with a consistent decrease in collagen within the fibrocartilage regions. This gradation ensures smooth mechanical stress distribution, improving the bonding strength, and decreasing the risk of fracture or rupture. Moreover, there is also a change in cell type and morphology along the interface. Enthesis fibrocartilage acts as a barrier to cell communication between ligament's fibroblasts and bone's osteocytes, as fibrocartilage is poorly vascularized and fibrochondrocytes do not express connexins and do not form gap junctions, therefore the intercellular communication occurs indirectly through cell-matrix interaction or soluble factors, these contribute to poor healing response at interface.

Tendons, conversely, have been shown to heal at the midsubstance when common suturing techniques and biologics are used for repair. Yet, tendon avulsions (rupture at the bone interface) require a repair technique similar to that of the ligament where bone tunnels are used to reattach the tendon to the bone. The enthesis for tendons varies slightly by skeletal attachment site but is similar to that for ligaments. It consists of gradients in mechanical properties, chemical properties and cell phenotypes, and is structurally continuous over multiple compositionally distinct regions. Fibrous entheses (bony or periosteal) are common in tendons that attach to the diaphysis of long bones and are characterized by dense fibrous connective tissue. Fibrocartilaginous entheses are common in tendons that attach at the epiphyses and apophyses and have four distinct regions that provide a structurally continuous gradient from bone to tendon: dense fibrous connective tissue, unmineralized fibrocartilage, mineralized fibrocartilage, bone.

Tendon and ligament are soft-tissues whose primary function is to stabilize and resist tensile loading. The interface of tendon/ligament to bone are specially adapted to allow for dissipation of stresses during loading. Current surgical repair/reconstruction techniques fail to adequately reproduce this interface and are prone to secondary failure at the interface.

Tissue engineering strategies to regenerate this interface have not yet succeeded at reproducing the gradients necessary to ensure proper stress distribution at the entheses. This is likely due to limitations in the manufacturing process.

3D bioprinting can be defined as the printing of biopolymers, biocompatible synthetic polymers, and high-concentration cell solutions. Typically, a 10 to 1000 μm resolution is required to form the internal structure of these tissue-like scaffolds, with higher-viscosity materials often providing structural support for the printed structure and lower-viscosity materials providing a suitable environment for maintaining cell viability and function. Laboratories across the world have designed and created 3D bioprinters to fabricate replacement human-scale tissues and organs, often with structural integrity and biological function similar to native tissues. These printed structures have been shown to be stable and amenable to revascularization, making them ideal for application in replacement of injured tissue. One tissue type that has not been adequately reproduced using 3D bioprinting alone includes musculoskeletal tissue subject to high tensile and compressive loads (ligament, tendon, cartilage, etc.).

As shown in FIG. 4, the electrospinning fabrication technique uses high voltage to create an electric field between a droplet of polymer solution (typically at the tip of a syringe needle (406)) and a conductive collector plate (490). The main forces acting on the polymer droplet are the electrostatic field and the electrostatic repulsion of charges. These forces are opposed by the surface tension of the droplet, and the viscoelastic forces of polymer. When electrostatic repulsion charges exceed the surface tension, stretching (i.e., elongating at very high strain rates) of the polymer droplet occurs, and a continuous fiber is ejected toward the collector plate, often in an uncontrolled fashion, except when the collector surface is modified to rapidly rotate such as in a drum, spindle, or mandrel configuration. Polymer solution viscosity, surface tension, electrical conductivity, and dielectric constant are key parameters for the electrospinning process controllable by solution selection and optimization. Applied voltage, flow rate of solution, collector material properties, diameter of the needle, and distance between needle and collector are other key parameters of the electrospinning process controllable by hardware setup, selection, and design. Conventional electrospinning techniques (5-35 kV, 2-30 cm needle to collector distance) can produce polymer fibers with diameters in the tens of nanometers to tens of micrometers size. When significantly reducing the voltage (<5 kV) and source to collector distance (<2 cm) in a near-field electrospinning configuration and allowing for a movable collector surface or movable applicator/needle, it is possible to enable a more controlled deposition of the polymer, typically at the expense of fiber diameter (hundreds of nanometers to hundreds of microns in size). Temperature, humidity, and pressure should also be considered when using this technique.

Traditionally, electrospinning processes can produce structures with excellent micro/nanostructural porosity, density and tensile strength, while lacking macroscale geometric control. These characteristics contrast to those of the 3D-bioprinting process that allows for excellent macroscale geometric control but is limited in the ability to produce structures resistant to high tensile loads.

### SUMMARY OF THE INVENTION

In one embodiment, the engineered hierarchical scaffold of the present invention may match the tensile and viscoelastic properties of the native ligament and provide a ligament/tendon-bone region that allows for the regeneration of a graded transition from soft to hard tissue for integration into existing structures.

In yet other embodiments, the present invention provides a scaffold with fiber alignment along the direction of applied tensile load while addressing all mechanical, biochemical and biophysical requirements for tissue growth.

In yet other embodiments, the present invention provides a scaffold fabricated by a combination of 3D bioprinting and aligned electrospinning process to allow for a functionally graded transition from soft tissue to bone.

In yet other embodiments, the present invention uses synthetic and/or natural polymers for electrospinning fibers that constitute the main component of the dense fibrous connective tissue (ligament, tendon, cartilage, meniscus, etc.) phase.

In yet other embodiments, the present invention provides a functional tissue scaffold that allows for regeneration of native tissue without the morbidity associated with harvesting of material from other regions of the body.

In yet other embodiments, the present invention provides a scaffold that introduces a hydrogel made from decellularized ligament/tendon/bone as polymer bioinks coupled with synthetic and/or natural electrospun polymers to promote cell adhesion and growth along the fibers.

In yet other embodiments, the present invention provides a hydrogel that may be reinforced with Hydroxyapatite (HAp) nanoparticles in the soft tissue to bone interface region to aid in providing the graded mechanical properties needed to transition from soft tissue to bone. HAp is a calcium phosphate mineral that constitutes the main material of bones.

In yet other embodiments, the present invention uses an optimization procedure to design an architecture for 3D printed bone and interface phases that will provide the graded structural properties needed to transition from soft tissue to bone.

In yet other embodiments, the present invention uses bioceramics and natural and/or synthetic polymers as bioinks for 3D printing the bone, interface (mineralized fibrocartilage and unmineralized fibrocartilage) phase of the scaffold.

In another embodiment, the present invention provides a method for regenerating a ligament comprising: a scaffold that is functionally-graded from the bone phase to the ligament phase. This can be extended to any other connective soft tissue (such as tendon, cartilage, meniscus, etc.) at bone insertion, and not limited only to ligaments.

In another embodiment, the present invention provides a device for regenerating a ligament comprising: a scaffold that is functionally-graded from the bone phase to the ligament phase. This can be extended to any other connective soft tissue (such as tendon, cartilage, meniscus, etc) at bone insertion, and not limited only to ligaments.

In another embodiment, the present invention provides a method and device wherein the scaffold includes hierarchical layers of 3D printed materials such as thermoplastics, bioceramics, and polymer bioinks (B) and electrospun fibers (F), alternated in a variety of thicknesses, patterns and architectures, optimized for anatomical region and mechanical properties of native tissue.

In another embodiment, the present invention provides a device and method wherein the 3D printed materials provide structural support for the bone and interface phases and support for cell viability, while fibers provide structural integrity for the ligament (soft tissue) and interface phases.

In another embodiment, the present invention provides a device and method wherein microstructure and mechanical properties are optimized for stem cell differentiation and proliferation. Moreover, it will aid fibroblast, osteoblast, chondroblast, and other cell proliferation for tissue regeneration with regional cellular population, density distribution and function of the native tissue.

In another embodiment, the present invention provides a device and method wherein multi-materials are used for functional gradation discretely or continuously. This may be achieved by multi-extrusion additive manufacturing (3D-printing) with varied concentration, mixtures and compounds of bioinks, and dispersed particles.

In another embodiment, the present invention provides a device and method wherein dispersed micro/nanoparticles concentrations are used to all extra levels of functional gradation, to alter microstructural, rheological and mechanical properties of bioinks.

In another embodiment, the present invention provides a device and method for regenerating a ligament comprising: an engineered hierarchical scaffold that matches the viscoelastic and mechanical properties of the native ligament and provides a ligament-bone region that allows for the regeneration of a graded transition from soft to hard tissue for integration into existing native soft and hard tissue.

In another embodiment, the present invention provides a device and method for regenerating a ligament comprising: a manufacturing technology or combination of technologies that allows for the fabrication of a hierarchical ligament-bone complex with fiber alignment along the direction of applied tensile load while addressing mechanical, biochemical and biophysical requirements for tissue regeneration.

In another embodiment, the present invention provides a device and method for regenerating a ligament comprising: a functional tissue scaffold that allows for regeneration of native tissue without the morbidity associated with harvesting of tissue from other regions of the body.

In another embodiment, the present invention provides a device and method for regenerating a ligament comprising: a scaffold that uses a hydrogel made from decellularized biomaterial as a bioink coupled with synthetic and/or natural electrospun polymer fibers.

In another embodiment, the present invention provides a device and method wherein the hydrogel is reinforced with Hydroxyapatite (HAp) particles in the ligament-bone interface region to provide the graded mechanical properties needed to transition from ligament to bone.

In another embodiment, the present invention provides a device and method wherein in order to replicate the biochemical and biophysical gradient at the ligament-bone interface, an additional mineral component of HAp may be included in the stepwise formation of the ligament-bone scaffold.

In another embodiment, the present invention provides a device and method wherein dual deposition of isolated decellularized ligament and/or bone and/or tendon and/or cartilage hydrogel and isolated HAp in solution is used to form a continuously graded transition region.

In another embodiment, the present invention provides a device and method for regenerating a ligament comprising: a scaffold that acts as a template for ligament and bone regeneration.

In another embodiment, the present invention provides a device and method wherein the scaffold is seeded with cells, and growth factors, and subjected to stimuli.

In another embodiment, the present invention provides a device and method wherein the scaffold cultured in-vitro and then implanted or implanted directly into the injured site where regeneration is induced in-vivo.

In another embodiment, the present invention provides a device and method wherein the scaffold has a functionally-graded characteristic that provides a gradual transition from the bone phase to ligament phase then back to the bone phase using polymers with a varying concentration of dispersed particles (i.e. Hydroxyapatite) and other necessary material modifiers.

In another embodiment, the present invention provides a device and method wherein the scaffold has a functionally-graded characteristic that provides a gradual transition from the bone phase to tendon, cartilage, meniscus, or other soft tissue interfacing with bone using polymers with a varying concentration of dispersed particles (i.e. Hydroxyapatite) and other necessary material modifiers.

In another embodiment, the present invention provides a device and method for regenerating a ligament comprising: bulk scaffolds that vary vertically and horizontally and are made from alternating layers of bioinks with optimized architecture at each phase and aligned or unaligned fibers.

In another embodiment, the present invention provides a device and method wherein the bioinks include biodegradable hydrogel or decellularized tissue and are tuned to serve as a viable extracellular matrix environment to support cell migration, growth, and proliferation.

In another embodiment, the present invention provides a device and method wherein the fibers are tuned (non-aligned vs. aligned orientation, porosity, fiber diameter, fiber spacing, length, etc.) to support high tensile loads such as those experienced by the native ligament.

In another embodiment, the present invention provides a device and method for regenerating a ligament comprising: a 3D bioprinter and electrospinner—in conventional and near-field configurations.

In another embodiment, the present invention provides a device and method that includes print heads and abstract material processing tools, including but not limited to, filament extruders, syringe pumps, electrospinner, router, etc. Multiple print heads may be provided for applying varied printing materials, bioinks, and electrospinning solutions that include but are not limited to molten polymers, cell-laden hydrogels, and polymer solutions.

In another embodiment, the present invention provides a device and method wherein the printer is controlled by one or more multi-axis motor controllers. Multiple motors may also be used: two for the X, Y build plate motion, one each for Z positioning of the abstract tools (3D print heads, electrospinner head, router, filament extrusion head, etc.). Furthermore, the material dispensing syringes (one for each print head, one for electrospinner, etc.) may be controlled using motors, pneumatic dispensers, or hydraulic dispensers.

In another embodiment, the present invention provides a device and method wherein the printer is controlled by one or more multi-axis motor controllers. Multiple motors may also be used: two motors can be used for X, Y positioning of the abstract tools and print heads and one motor can be used for Z positioning of the build plate. Furthermore, the material dispensing syringes (one for each print head, one for electrospinner, etc.) may be controlled using motors, pneumatic dispensers, or hydraulic dispensers.

In another embodiment, the present invention provides a device and method that are adaptable for additional print heads or abstract tools as necessary by application and properties of printing material.

In another embodiment, the present invention provides a device and method further including an X-Y stage that enables positioning and adjusting of the build plate under the 3D bioprinter or electrospinner deposition heads, respectively and successively, and multiple Z axes vertically mounted to the frame of the printer, allowing for height control (i.e. material deposition thickness and build plate out-of-plane patterns and architectures) of the abstract tools (print heads, electrospinner head, router, filament extruder head, etc.).

In another embodiment, the present invention provides a device and method further including an X-Y stage enables positioning and adjusting of the deposition heads and a Z axis that allows for height control of the build plate.

In another embodiment, the present invention provides a device and method further including a build plate designed as a surface for printed material, containing a grounded collector for the electrospinner of the system. The grounded collector is made from high conductivity materials including, but not limited to, copper, steel, indium tin oxide, and/or carbon.

In another embodiment, the present invention provides a device and method wherein the build plate is a layered system of electrical and thermal conductive and insulating materials supported by leveling screws. One layer or more may include a heated element. An air gap may also be provided between the build plate and linear stage configured to reduce unwanted conductivity from the electrified collector plate during electrospinning.

In another embodiment, a plurality of syringe pumps may be included. The electrospinner may also consist of a syringe, metallic needle, conductive plates of the collector, and one or more variable high-voltage power supplies.

In another embodiment, the present invention provides a device and method wherein one lead of the power supply clamps to the collector of the build plate, and the other lead connects directly to the needle of the mounted syringe, as voltage is applied, material fibers collect on the surface of the collector.

In another embodiment, the present invention provides a device and method further including one or more polymer filament extruders to allow for deposition of thermoplastic and/or thermoset polymers to print custom patterns, architectures and multi-materials structures.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1A illustrates a functional tissue scaffold that allows for regeneration of native tissue subject to high tensile loads for an embodiment of the present invention.

FIG. 1B illustrates a section that may be repeated to form a functional tissue scaffold that allows for regeneration of native tissue subject to high tensile loads for an embodiment of the present invention.

FIG. 2A illustrates a functional tissue scaffold that allows for regeneration of native tissue subject to high tensile loads for another embodiment of the present invention.

FIG. 2B illustrates a section that may be repeated to form a functional tissue scaffold that allows for regeneration of native tissue subject to high tensile loads for an embodiment of the present invention.

FIG. 3 illustrates a functional tissue scaffold that allows for regeneration of native tissue subject to high compressive loads for an embodiment of the present invention.

FIG. 4 is a schematic of a 3D bioprinting and electrospinning dual apparatus.

FIG. 5A is a schematic of a 3D bioprinting and electrospinning dual apparatus in an alternate configuration. Note that the top plate and vertical front supports have been removed from the image so that internal structure can be viewed.

FIG. 5B is a schematic of a 3D bioprinting and electrospinning dual apparatus in the alternate configuration of FIG. 5A, but top plate, vertical front supports, side walls, door, and windows are included.

FIG. 6A illustrates how unaligned fibers may be created for the scaffolds of the present invention using electrospinning.

FIG. 6B illustrates how aligned fibers may be created for the scaffolds of the present invention using electrospinning.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

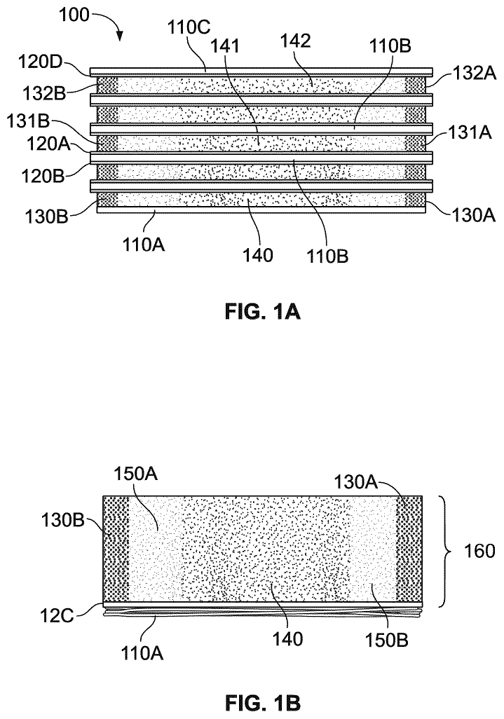

A representative schematic of a scaffold 100 for an embodiment of the present invention is shown in FIGS. 1A and 1B. One or more electrospun fibers 110A, 110B and 110C may be made from a plurality of synthetic and/or natural polymer. Layers 110A and 110C, as well as others in the scaffold, form the top and bottom of the scaffold with the other layers sandwiched in-between to form a composite of layers of different electrospun and/or 3D printed materials. The fibers may be a composite of one or more collagen-rich layers (120A-120D) to improve adhesion to the other layers and facilitate cell activity.

One or more 3D printed polymer sections (140-142) represent the soft tissue phase of the scaffold and are made using biomaterials that are mechanically, chemically, and histologically similar to the soft tissue targeted for regeneration. One or more 3D printed sections (130A, 130B, 131A, 131B, 131A and 132B) represent the bone phase of the scaffold and are made using biomaterials that are mechanically, chemically, and histologically similar to bone. Also provided are one or more functionally graded interfaces 150A and 150B. These interfaces will be materially and architecturally graded to transition from the soft-tissue (ligament, tendon, cartilage, etc.) phase (140) to the bone phase on each end (130A and 130B). As shown in FIG. 1B, layers or sections (110A, 120C, 130A, 130B, 150A, 150B, and 140) form a section 160 that may be repeated to form a plurality of sections as shown in FIG. 1A. In other aspects, layers 120A-120D include collagen-rich PLGA to improve adhesion to the hydrogel and facilitate cell attachment. As shown, collagen-rich PLGA layer 120A and 120B are located on both sides of layer 110B. In yet other aspects, fibers 110A, 110B and 110C may be made from polylactic-co-glycolic acid (PLGA).

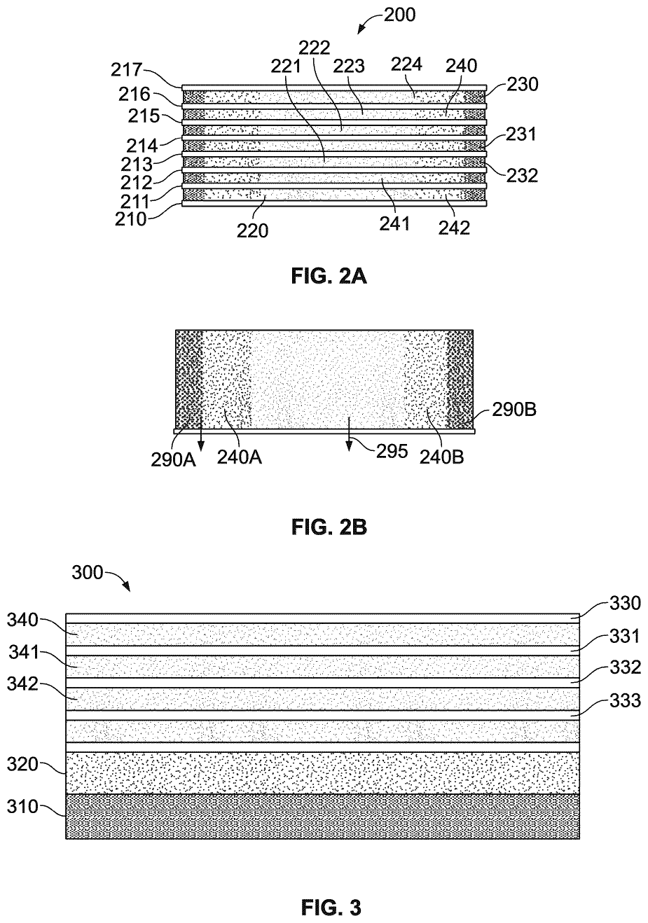

FIGS. 2A and 2B illustrate another embodiment of the present invention. For this embodiment, two or more different materials may be used to fabricate the soft tissue-bone scaffold (200). These materials may form: i) one or more electrospun fibers (210-217), ii) one or more coatings to promote cell adhesion, iii) one or more 3D printed soft tissue phase layers (220-224), iv) one or more 3D printed bone tissue phase layers (230-232, 290A, and 290B) and v) one or more functionally graded soft tissue-bone interfaces (240-242). The fibrous phase of the scaffold may be conventionally or near-field electrospun from a solution of synthetic and/or natural polymers.

3D printed layers (220-224), as well as the others shown, may be made from decellularized bone, hydroxyapatite (Hap), and/or other functional nano/micro particles in a synthetic and/or natural polymer solution.

Bone-soft tissue interfaces (240-242) may be considered as a third phase. These layers may be printed in a functionally graded manner near the electrospun fiber ends to form a bone-ligament-bone scaffold.

In a preferred embodiment, layers 220-224 may be hydrogels made from a decellularized ligament-derived hydrogel (LDH), which is an optimal milieu of macromolecules conducive to native material regeneration. Also, ligament-bone interfaces 230-232 may be made of LDH reinforced with nano-HAp. These layers may be configured in a functionally graded manner at the electrospun fiber ends to form a bone-ligament-bone scaffold.

In other embodiments, the fibers extend parallel to bone, gradient and soft-tissue phases. Alternately, the fibers extend through the bone, gradient, and soft-tissue phases.

The fibrous material phase may be formed through aligned electrospinning by adjusting the parameters of the system in a near-field electrospinning configuration (<2 cm needle to collector distance, <5 kV, moving collector and/or tool head). The ratio of synthetic and/or natural polymers, fibers, and nano/micro particle constituents governs the mechanical properties, degradation rate, and hydrophobicity of the scaffold.

While the electrospun fibers addresses the biomechanical properties of the native ligament, it is necessary to provide an environment conducive to cellular attachment, growth, migration, and proliferation. This may be achieved through addition of a polymer matrix deposited in an alternating fiber-polymer-fiber fashion via 3D bioprinting.

In order to replicate the biochemical and biophysical gradient at the ligament-bone interface, an additional mineral component of decellularized bone, Hap, or other biocompatible nano/micro particles may be included in the stepwise formation of the scaffold. HAp, an inorganic salt, has excellent osteoconductivity and biocompatibility and has been shown to induce osteoblast proliferation.

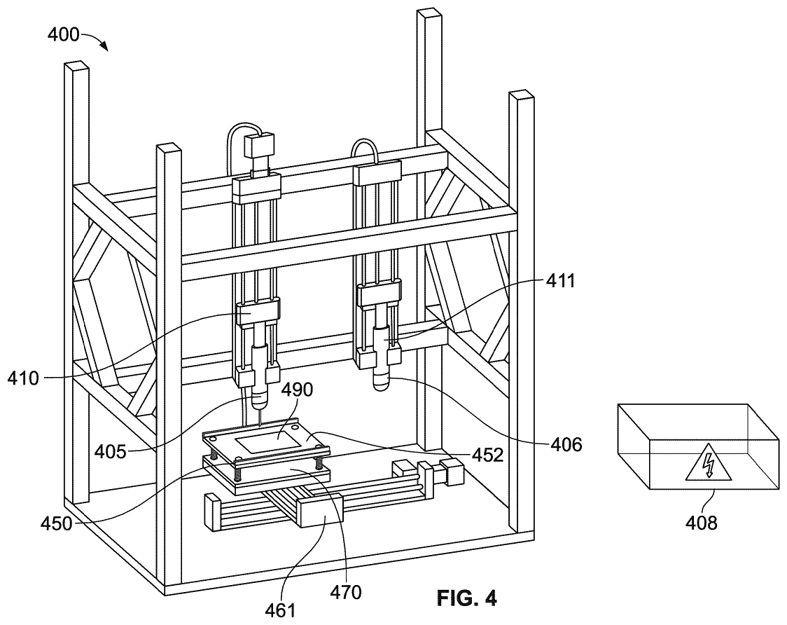

FIG. 3 illustrates another embodiment of the scaffold (300). In the instance where cartilage, meniscus, or other similar interface tissue is fabricated, the direction of the functionally-graded regions may change. Unlike FIGS. 1 and 2 depicting bone-ligament-bone scaffolds showing the bone and interface regions at the left and right ends of the scaffold, this embodiment shows the bone (310) at the bottom, moving vertically through the interface region (320), and further vertically showing the alternating 3D printing (340-342)/electrospun fiber layers (330-333). This is the preferred embodiment for other bone —soft tissue scaffolds such as cartilage and meniscus where the primary mode of external loading is compressive.

Electrospinners require a high voltage power supply (up to 30 kV), a syringe with a needle and a conducting collector. An electrode is placed in the syringe with the polymer solution or attached to the needle to provide a uniform charge to the liquid, and the other electrode is attached to the collector plate. The polymer experiences electrostatic repulsion on its surface due to the applied charge and is also attracted to the collector plate via a Coulombic attraction. These forces work against the surface tension of the polymer solution to create a cone of fluid, known as a Taylor Cone, at the tip of the metal syringe. Above a threshold voltage, the electrostatic forces are enough to overcome the surface tension and a thin jet of fluid is ejected toward the collector plate. This technique has been used on over 50 polymers to create fibers with diameters ranging from <3 nm to over 1 μm and lengths up to several kilometers.

This jet of polymer solution typically displays unstable fluid flow and will therefore deposit onto the collector plate with a random orientation. For this reason, many techniques have been developed in an attempt to align the fibers during collection.

The diameter of the fibers can be controlled to some degree by varying parameters of the system, most notably the viscosity of the solution, distance from needle to collector, strength of the applied electric field, electrical conductivity of the collector surface, and feeding rate for the solution. Varying the concentration of polymer in the solution can alter the viscosity of the solution. The diameter of the fiber increases with the viscosity of the solution, though at very low viscosities defects such as beads can appear along the fibers. Increasing the electrical conductivity helps to significantly reduce the diameter of the fibers possible without defects. The fiber diameter also increases with feeding rate. However, the correlation between electric field strength and fiber diameter is not well established. Through manipulation of the above parameters, the diameter of the fibers may be tailored for use in specific applications. In a preferred embodiment of the present invention, aligned electrospun fibers with diameters between 1 and 15 microns may be used to facilitate MSC differentiation into the ligament lineage and associated with spindle-shaped morphology in human ligament fibroblasts.

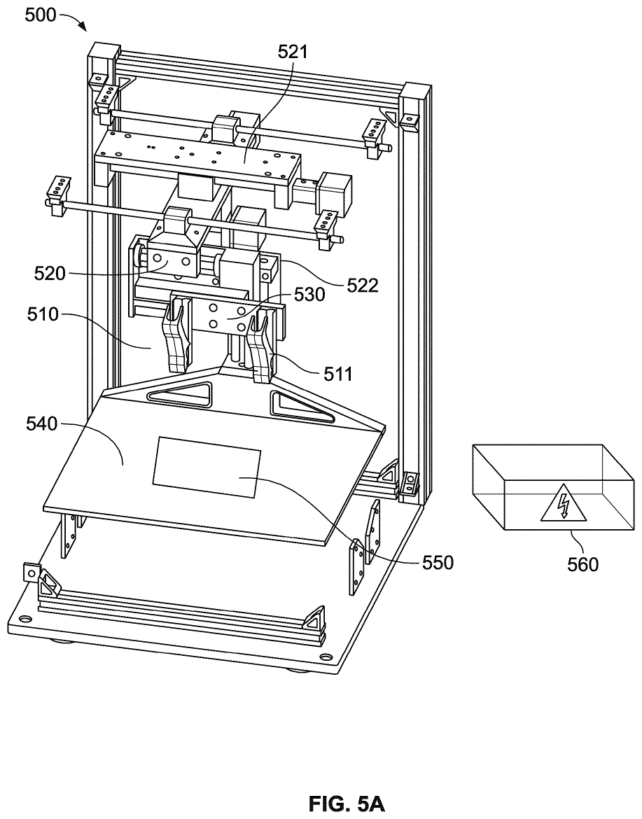

In yet another embodiment of the present invention as shown in FIG. 4, a hybrid 3D bioprinter and electrospinner system (400) is provided. The system (400) is configured to accommodate the polymer bioinks, filament extruders, and electrospinner polymers for composite scaffold formation. Motion of the tool heads (405 and 406) is controlled using rails (410-411) that allow for Z-axis control of carriages that engage the rails supporting the tool heads. X-Y axis motion of the build plate (450) is controlled by perpendicularly positioned stepper motors and linear screw drives, which carries the x-y module (460-461). An air gap (470) is included between the build plate/collector and the non-conductive support engaging the linear rails to limit interference from the high voltage power supply (480). The collector (490) is positioned centrally in/on the build plate. One electrical lead is connected from the power supply (480) to the collector (490) and the other is connected from the power supply to the needle of the electrospinning syringe.

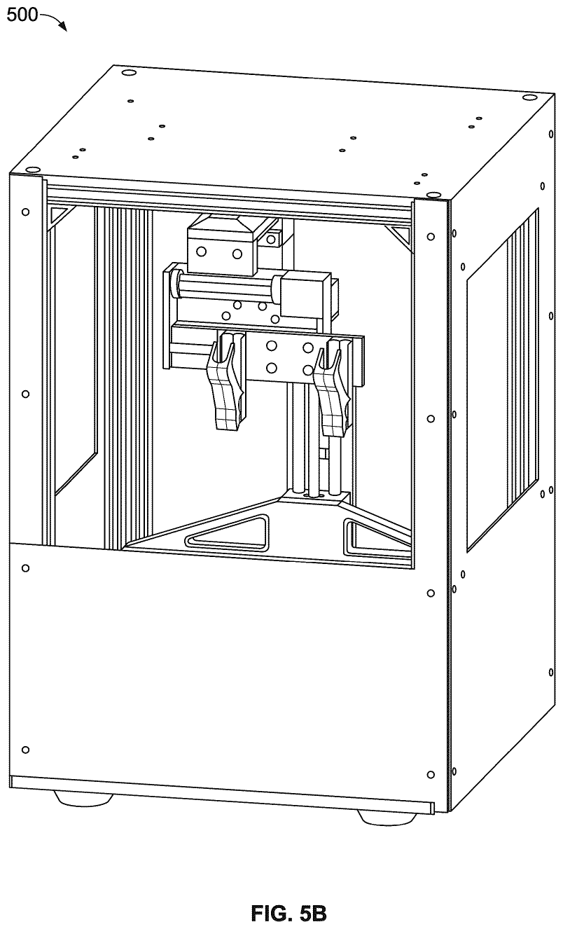

In yet another embodiment of the present invention as shown in FIGS. 5A and 5B, a hybrid 3D bioprinter and electrospinner system (500) is provided. The system (500) is configured to accommodate the polymer bioinks, filament extruders, and electrospinner polymers for composite scaffold formation. Motion of the attached tool/print heads (510 and 511) is controlled using perpendicularly positioned rails 520 and 521 that allows for X-Y axis control of the carriage (530) that engages the tool heads. Z axis motion of the build plate/collector (540) is controlled by a stepper motor and linear screw drive (522, partially hidden), which carries the Z carriage engaging the build plate. The collector (550) is positioned centrally in/on the build plate. One electrical lead is connected from the power supply (560) to the collector (550) and the other is connected from the power supply to the needle of the electrospinning syringe. FIG. 5B shows the system (500) of FIG. 5A with outer walls and windows used to control temperature, humidity, and pressure within the system. Additionally, the lower region of the internal structure of the system will contain a system for UV polymerization of polymers. The enclosed system will shield the user from exposure to UV radiation.

The system is controlled by electronics that provide control to the X, Y, and Z rail positioners. The syringe toolheads may be dispensed using motors, pneumatic dispensers, hydraulic dispensers, or other similar mechanism for dispensing. One or more high voltage power supplies (1-35 kV) provide the voltage source for conventional and near-field electrospinning. The electronics may control syringe dispensing, the filament extruder, the power supplies, and other onboard accessories that may be added to the system including but not limited to an ultraviolet (UV) light chamber for polymerization, the other abstract tool heads, and devices for controlling temperature, humidity, and pressure.

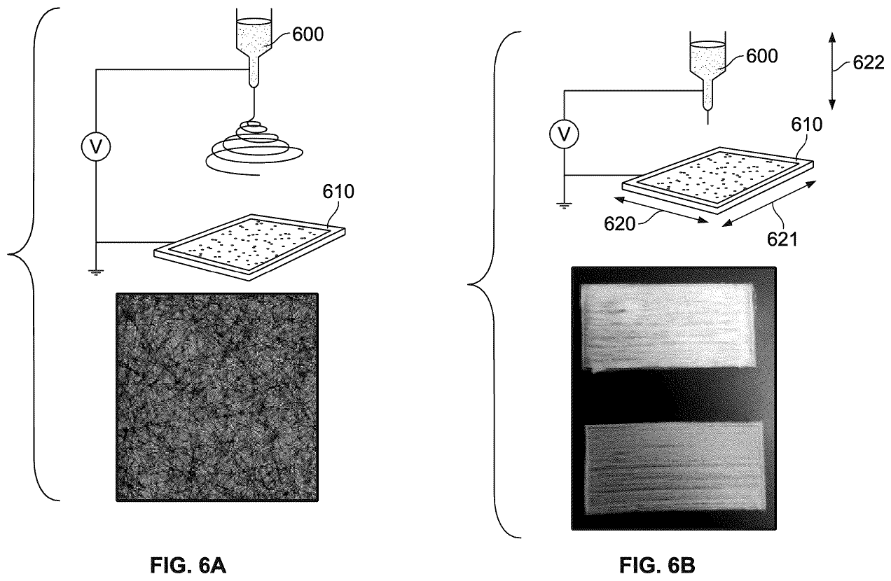

FIG. 6A illustrates how unaligned fibers may be created for the scaffolds of the present invention using the conventional electrospinning technique (>2 cm needle to collector distance, >5 kV) from source 600 onto collector 610. FIG. 6B illustrates how aligned fibers may be created for the scaffolds of the present invention using near-field electrospinning (<2 cm needle to collector distance, <5 kV, and X, Y, Z positioning of the syringe/needle and/or the collector) from source 600 onto collector 610. The figure shows arrows representing FIG. 4 configuration of linear rails (620, 621, and 622) moving the build plate/collector in the X-Y axes and the print heads in the Z axis.

In a preferred embodiment, the present invention takes into consideration the fact that ligament healing is not efficient and often results in weak scar tissue that influences the graft stability in the bone tunnel. To address this problem, the present invention provides a tissue-engineering strategy that provides functionally-graded scaffolds from soft to hard tissue. As shown in FIG. 2B, the functionally-graded characteristic may allow a gradual transition from the bone phase 290A to ligament (or other soft tissue) phase 295 then back to the bone 290B phase using natural and/or synthetic polymers with a varying concentration of micro/nanoparticles (i.e. Hydroxyapatite) and other necessary material modifiers. The bulk scaffolds may vary vertically made from alternating layers of bioinks (synthetic and/or natural polymers are 3D printed with optimized architecture at each phase) and fibers (i.e. electrospun collagen and/or natural or synthetic biopolymers). The bioinks which may also include hydrogel biomaterials or decellularized tissue may be tuned to serve as a viable extracellular matrix environment to support cell migration, growth, and proliferation. The fibers may be tuned (non-aligned vs. aligned orientation, porosity, fiber diameter, fiber spacing, length, etc.) to support high tensile loads such as those experienced by the native ligament; these fibers provide most of the mechanical stability and strength of the bulk scaffold. Multiscale material and structural optimization can be used to control microstructure, mechanical properties, and biodegradation rates of the scaffold. The 3D printed aspect of this approach enables macroscale structural features of the replacement tissue to match that of the targeted native tissue.

In other embodiments, the present invention fabricates 3D hierarchical, functionally-graded scaffolds and tunes the biodegraded scaffold mechanical properties to comply with the ligament regeneration rate to maintain mechanical properties at a satisfactory level during the tissue growth period and thereafter. The scaffold may act as a template for ligament regeneration and is typically seeded with cells, and growth factors, and subjected to stimuli. In other embodiments, the scaffolds may be either cultured in-vitro and then implanted or implanted directly into the injured site where regeneration is induced in-vivo.

In yet another embodiment, the present invention provides a modular 3D Bioprinter and Electrospinner system for targeted fabrication of scaffolds for tissue engineering and other biomedical applications. The hybrid system aims to merge the positive aspects of each technology. In a preferred embodiment, the present invention is particularly useful for creation of tissue scaffolds which are configured to resist high tensile loads (i.e. ligament, tendon, bone-ligament interface, etc.).

In some aspects, the present invention is designed using a control system similar to 3D printers, but with modification to input software files to control for alternating deposition of materials from the 3D printing side and the electrospinner side of the system. This allows for the creation of hierarchical scaffolds with alternating layers of 3D printed bioinks, extruded polymer filaments, and electrospun materials. The print heads can be tuned to deliver bioinks in the form of synthetic or natural polymers including hydrogels or decellularized tissue solutions with various concentrations of macro/micro/nanoscale particles for targeted deposition of materials depending on the tissue type. This deposition is meant to serve as an optimal microenvironment for encouraging cell growth, migration, proliferation and can include growth factors and cells in solution as a bioink that can be deposited from one or more print heads (or abstract tools) in any defined architecture.

In yet other embodiments, the present invention may be comprised of one or more of the following components depending on the desired application and use: Microcontroller: The system is controlled by a multi-axis stepper motor controller with flexible software configuration. The system is designed to be extensible, adapting to various hardware and software configurations. In one embodiment, the system uses motors: for X, Y, and/or Z positioning of the abstract tools (3D print heads, electrospinner head, router, filament extrusion head, etc.) and build plate/collector, and one each to drive custom syringe pumps (one for each print head, one for electrospinner, etc.), filament extruder, and other abstract tools. The system is adaptable for additional print heads or abstract tools as necessary by application. The other inputs of the controller could also run several systems for temperature, humidity, UV polymerization, heated beds, and other system accessories.

Linear Stages: High precision linear stages are used for X, Y, and Z axis movement to control for resolution, accuracy and repeatability. Each stage may be fitted with stepper motors. The leadscrew configuration of the linear stages allows for precise movement. The X stage enables positioning of the build plate/collector under the 3D bioprinter or electrospinner deposition heads or the deposition heads above the build plate/collector. The Y stage is mounted orthogonally to the X stage for front-to-back positioning of either the deposition heads or the build plate/collector. The Z axis may be vertically mounted to the frame of the system, allowing for height control of the deposition heads or build plate/collector

Build plate: The build plate (print bed) is designed as a surface for printed material and as an electrical conductor for the electrospinner of the system. The build plate is a layered system of conductive and electrically-insulating materials supported by leveling screws. The electrically-conductive layer can be made using copper, steel, indium tin oxide, and any other conductive material that enables high resolution deposition and control of electrospun fibers.

To help reduce unwanted conductivity from the electrified collector during electrospinning, an air gap is formed between the build plate and linear stage. Additionally, the build plate may be attached to the linear stage using nylon screws or other non- or minimally-conductive material to further reduce unwanted electrical charge throughout the system.

Another embodiment of the present invention as shown in FIG. 4, includes a build plate (450) that includes a non-conducting material (452) that allows for deposition of materials from the tool heads, but limits conductivity for the electrospinner outside of the conductive collector region. The new collector surface for electrospinning are one or more high-conductive sections (490) which may be in the shape of raised bars, inset plates or coatings in the build plate, or other geometries. The one or more conductive sections (490) are placed on or in non-conductive surface (452) to allow for fiber deposition from the electrospinner side of the system. The voltage and needle to collector distance determines the type of fiber deposition (uncontrolled or aligned) to be deposited on the collector.

Syringe Dispensers/3D printing tool heads: The extrusion system consists of two or more syringe holders that may be constructed using custom or commercially available off-the-shelf hardware (rails, bolts, nuts, etc.). The fixture that holds the syringe allows access to slide bearings and improves the grip on the plunger of the syringe during deposition. Each syringe may be dispensed using motors, pneumatic dispensers, hydraulic dispensers, or any similar dispensing system.

Electrospinner tool head: The electrospinner tool head consists of a syringe, steel syringe tip/needle, the conductive collector of the build plate, and one or more 1 to 35-kV variable high-voltage power supplies. The power supply has positive and negative leads the attach to the needle of the syringe and to the collector plate to apply a voltage from needle to collector plate during deposition. The syringe may be dispensed using motors, pneumatic dispensers, hydraulic dispensers, or any similar dispensing system.

In operation, one lead of the power supply clamps directly to the conductive collector plate, and the other lead connects directly to the steel needle of the mounted syringe. As voltage is increased, an electric field is created between the syringe needle and collector. Solution exiting the syringe becomes charged and quickly collects on the surface of the charged collector plate.

3D-Modeling Software: Any commercially available 3D modeling software can be used to create the input files. The current hardware configuration is not limited to any 3D modeling software.

Slicing Software: The current configuration of the system works with any commercially available slicing software. For more complex scaffold geometries and to enable alternating deposition of 3D printed and electrospun fibers it is necessary to use the custom slicing software or modify the source code of commercially available software. It is highly recommended that a more sophisticated software that allows for voxelization of the scaffold (discretizing the structure into elements to define elemental material properties) be used.

Filament Extruders: The system contains one or more polymer filament extruders to allow for deposition of thermoplastic polymers to print custom bioarchitectures and multiphase scaffolds.

Extra features: The system uses UV light for photopolymerization or crosslinking of polymers upon deposition from the tool heads. A Peltier cooler can be used to reduce the temperature of the materials in each syringe prior to deposition if necessary by application. A flexible heat bed can be used to increase the temperature of the materials in each syringe prior to deposition if necessary by application. The system uses additional temperature, humidity, and pressure controlling hardware to maintain a suitable environment for deposition.

In other embodiments, the present invention provides a method for fabricating a device for regenerating musculoskeletal tissue comprising the steps of: creating scaffold or other structure having an aligned fiber layer adapted to provide mechanical integrity to the scaffold in the form of increased tensile and compressive resistance; creating an interface layer comprised of one or more bone phases that is adapted to resemble the biophysical and biochemical structure of bone, one or more soft tissue phases adapted to resemble the biophysical and biochemical structure of the soft tissue to be regenerated, and one or more gradient phases adapted to resemble the biophysical and biochemical interface between the bone and said soft tissue; each of the phases created using one or more materials; and repeating the above steps as needed. In other aspects, the aligned fiber layer is created by near-field electrospinning and the interface layer is created by 3D-printing. Alternately, the aligned fiber layer is created by the use of a print head and print bed configured to suppress the formation of a Taylor cone. In other aspects, the Taylor cone is suppressed by applying a voltage to the print head and print bed. The Taylor cone may also be suppressed by applying a voltage to the print head and print bed and separating the print head and print bed a sufficient distance.

A technique for near-field electrospinning may also be used by using the following parameters: the needle to collector distance is <2 cm, the voltage between the needle and the collector plate is <5 kV, and the collector and/or the needle can translate in X and Y directions, while the other moves in the Z direction.

In other embodiments, the present invention provides a system for 3D printing and electrospinning materials on the same build platform with a single control system by defining the parameters for each deposition. The system may include a 3D printer with one or more tool heads that are used for electrospinning and the build platform can move in the X, Y and/or Z directions. Also, the one or more tool heads can move in the X, Y, and/or Z directions. The system may also include tool heads which can be one or more of the following: syringes w/needles and filament extruders (hot ends) which can be controlled by one or more of the following: pneumatic pumps, hydraulic pumps, and motors. In other aspects, a high voltage power supply is connected to the collector plate and to the needle or filament extruder to create a circuit between the two objects allowing for electrospinning. Near-field electrospinning allows for highly aligned deposition of low-microscale diameter fibers. Melt electrospinning is performed using the following parameters: filament extruder to plate collector distance <2 cm, voltage <10 kV, moving build plate or tool head. Melt electrospinning allows for highly aligned deposition of mid-to-high microscale diameter fibers. The system may also include a sub-chamber containing ultraviolet (UV) lights that enables polymerization of said 3D printed or electrospun materials. The collector plate can be made from one or more of the following materials: steel, silver, aluminum, copper, carbon, silicon, indium tin oxide.

While the foregoing written description enables one of ordinary skill to make and use what is considered presently to be the best mode thereof, those of ordinary skill may understand and appreciate the existence of variations, combinations, and equivalents of the specific embodiment, method, and examples herein. The disclosure should therefore not be limited by the above described embodiments, methods, and examples, but by all embodiments and methods within the scope and spirit of the disclosure.

What is claimed is:

1. A device for regenerating musculoskeletal tissue comprising:
a scaffold comprised of a plurality of layers; and
at least one of said layers is comprised of fibers adapted to provide mechanical integrity to the scaffold in the form of increased tensile and compressive resistance and one or more layers are comprised of a biocompatible material adapted to provide mechanical integrity and to provide a suitable biochemical environment in all phases.

2. The device of claim 1 wherein said biocompatible material is comprised of one or more from the group comprising: bioceramics, polymers, and hydrogels.

3. The device of claim 2 wherein said biocompatible material is reinforced with one or more of the following: Hydroxyapatite (HAp) nanoparticles, decellularized ligament, decellularized tendon, decellularized bone, other decellularized soft tissue, and appropriate growth factors for the cells.

4. The device of claim 2 wherein said bioceramics include one or more of the following: calcium sulfate, hydroxyapatite, tricalcium phosphate-based materials, tricalcium phosphate/hydroxyapatite biphasic materials, alumina ceramics, zirconia ceramics, bioactive glass, bioactive glass-ceramics, calcium phosphate, and porcelains.

5. The device of claim 2 wherein said polymers include one or more of the following: proteins, carbohydrates, amino acids, peptides, polylactides (PLA), polyglycolides (PGA), polylactic-co-glycolic acid (PLGA), polyethylenes (PE), polycaprolactone (PCL), polyurethanes (PU), polyphosphazenes, polyanhydrides, polyacetals, polyphosphoesters, polyorthoesters, polyhydroxyalkanoates, polypropylene fumarate (PPF), polycarbonates, polyamides, and combinations of said polymers.

6. The device of claim 2 wherein said hydrogels include one or more of the following: thermo-responsive materials such as poly(N-isopropylacrylmide) (NIPAAm), pH-responsive materials such as poly(methyl methacrylate) (PMMA), polyethers, alginate, polyethylene glycol (PEG), polyethylene glycol-diacrylate (PEG-DA), polysaccharides such as cellulose, gelatin, and poloxamers.

7. The device of claim 1 wherein said fibers are comprised of one or more biocompatible materials.

8. The device of claim 7 wherein said biocompatible materials are comprised of one or more of the polymers of claim 5.

9. The device of claim 1 wherein said scaffold has a bone phase adapted to resemble the biophysical and biochemical structure of bone, a soft tissue phase adapted to resemble the biophysical and biochemical structure of the soft tissue to be regenerated, and a gradient phase adapted to resemble the biophysical and biochemical interface between the bone and said soft tissue.

10. The device of claim 11 wherein said bone phase, said gradient phase and said soft tissue phase are comprised of different materials.

11. The device of claim 7 wherein said fibers extend parallel to said bone, gradient and soft-tissue phases.

12. The device of claim 7 wherein said fibers extend through said bone, gradient, and soft-tissue phases.

13. The device of claim 11 wherein said bone phase, said gradient phase and said soft tissue phase are comprised of different materials of claim 2.

14. The device of claim 11 wherein said bone phase, said gradient phase and said soft tissue phase are comprised of different concentrations of the same materials of claim 2.

15. The device of claim 12 wherein said bone phases, said gradient phases and said soft tissue phases are comprised of different materials of claim 2.

16. The device of claim 12 wherein said bone phases, said gradient phases and said soft tissue phases are comprised of different concentrations of the same materials of claim 2.

17. A method of fabricating a device for regenerating musculoskeletal tissue comprising the steps of:
(a) creating a scaffold having an aligned fiber layer, said fiber layer adapted to provide mechanical integrity to the scaffold in the form of increased tensile and compressive resistance;
(b) creating an interface layer, said interface layer comprised of one or more bone phases wherein said bone phase is adapted to resemble the biophysical and biochemical structure of bone, one or more soft tissue phases adapted to resemble the biophysical and biochemical structure of the soft tissue to be regenerated, and one or more gradient phases adapted to resemble the biophysical and biochemical interface between the bone and said soft tissue;
(c) each of said phases created using one or more materials; and
(d) repeating steps (a)-(c) as needed.

18. The method of claim 17 wherein said aligned fiber layer is created by near-field electrospinning and said interface layer is created by 3D-printing.

19. The method of claim 18 wherein said near-field electrospinning technique uses the following parameters: providing a needle and collector, the needle to collector distance is <2 cm, the voltage between the needle and the collector plate is <5 kV, and the collector and/or the needle can translate in X and Y directions, while the other moves in the Z direction.

20. The method of claim 18 wherein the device is a scaffold having at least one bone phase, at least one gradient phase and at least one soft tissue phase.

21. The method of claim 18 wherein the device is a scaffold having a bone phase, a gradient phase, a soft tissue phase, a gradient phase and a bone phase.

22. The method of claim 21 wherein fibers extend through said phases.

23. The method of claim 20 wherein said fibers extend parallel to said bone phase and the gradient phase.

24. The method of claim 18 wherein said fibers are made from one or more polymers of claim 5.

25. The method of claim 20 wherein said bone phase is made from one or more fibers of claim 7 and one or more polymers of claim 5.

26. The method of claim 20 wherein said bone phase is made from one or more fibers of claim 7 and one or more bioceramics of claim 4.

27. The method of claim 20 wherein said bone phase is made from one or more fibers of claim 7, one or more bioceramics of claim 4, and one or more polymers of claim 5.

28. The method of claim 20 wherein said gradient phase is made from one or more fibers of claim 7 and one or more polymers of claim 5.

29. The method of claim 20 wherein said gradient phase is made from one or more fibers of claim 7 and one or more bioceramics of claim 4.

30. The method of claim 20 wherein said gradient phase is made from one or more fibers of claim 7, one or more bioceramics of claim 4, and one or more polymers of claim 5.

31. The method of claim 20 wherein said soft tissue phase is made from one or more fibers of claim 7.

32. The method of claim 20 wherein said soft tissue phase is made from one or more fibers of claim 7 and one or more hydrogels of claim 6.

33. The method of claim 20 wherein said soft tissue phase is made from one or more fibers of claim 7 and one or more polymers of claim 5.

34. The method of claim 20 wherein said soft tissue phase is made from one or more fibers of claim 7, one or more hydrogels of claim 6, and one or more polymers of claim 5.

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. The method of claim 17 wherein said fiber layer is created by the use of a print head and print bed configured to suppress the formation of a Taylor cone.

56. The method of claim 55 wherein said fiber layer is created by applying a voltage to the print head and print bed to suppress the formation of a Taylor cone

57. The method of claim 56 wherein said fiber layer is created applying a voltage to the print head and print bed and separating the print head and print bed a sufficient distance to suppress the formation of a Taylor cone
